(19) United States
(12) Patent Application Publication
(10) Pub. No.: US 2009/0032023 A1
(43) Pub. Date: Feb. 5, 2009

(54) CLOSED REVERSIBLE BREATHING APPARATUS HAVING A METAL ORGANIC FRAMEWORK

(75) Inventors: Jorg Pastre, Bensheim (DE); Ulrich Muller, Neustadt (DE); Markus Schubert, Ludwigshafen (DE); Christoph Kiener, Weisenheim am Sand (DE); Friedhelm Teich, Neckarhausen (DE); Frank Poplow, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/282,289

(22) PCT Filed: Feb. 26, 2007
(86) PCT No.: PCT/EP07/51788

§ 371 Date: Sep. 18, 2008

(30) Foreign Application Priority Data
Mar. 9, 2006 (EP) 06110880.9

(51) Int. Cl. A61M 16/22; B01J 49/00

(52) U.S. Cl. 128/205.28; 210/673

(57) ABSTRACT

The present invention relates to methods for removing carbon dioxide and, if appropriate, water from breathing air in closed or partially closed systems using a porous metal-organic framework material, such systems having at least one breathing apparatus and also their use and methods for regenerating the porous metal-organic framework material.

The present invention relates to methods for removing carbon dioxide and, if appropriate, water from breathing air in closed or partially closed systems using a porous metal-organic framework material, such systems having at least one breathing apparatus and also their use and methods for regenerating the porous metal-organic framework material.

In closed or partially closed systems it is necessary that owing to the limited supply of oxygen this must be replenished if, for example, a person wishes to remain in this system for longer than the oxygen supply which is provided by the volume of the system would permit.

Air or oxygen is generally supplied in this case by corresponding pressure vessels, such as, for example, pressure cylinders.

For instance it is necessary, for example, during diving that the diver, in addition to a diving mask, also carries in conjunction oxygen cylinders if the diver wishes to remain for a relatively long time under water.

Customarily in this case oxygen is supplied to the diver via a mouthpiece from the pressure cylinder, which the diver can breath. The expired air is released to the surrounding water. By this means the diver can remain under water for longer than the air volume of the diving mask would provide.

Nevertheless, the time spent below water is restricted for the diver by the volume of the pressure cylinder. A further possibility for optimization and, in association, a prolongation of the time spent below water is additionally using an adsorbent which is suitable for removing from the air the carbon dioxide present in the expired air in such a manner that the air having the remaining oxygen can be again provided for breathing.

Such systems having adsorbents are known in the prior art. Examples of these are described in EP-A 0 782 953, DE-A 197 167 49 and also DE-A 198 16 373.

The adsorbents described in the prior art which can be used comprise different materials.

In GB-A 1 438 757, for example, use is made of a soda lime bed for a diving apparatus.

WO-A 01/83294 describes, for example, a breathing apparatus, in which the carbon dioxide absorber is said to be able to be reactivated by heat or reduced carbon dioxide pressure. An example of such an absorber mentioned is calcium hydroxide.

DE-A3303420 describes methods and devices for purifying breathing air from CO₂, molecular sieves acting as adsorbers which can be regenerated by a pressure-swing method.

Finally, special adsorbents are described in EP-A 1 155 728. These are amino-methylated bead polymers.

Despite these numerous adsorbents proposed in the prior art, there is still a requirement to provide further optimized adsorbents for removing carbon dioxide and, if appropriate, water from the breathing air.

An object of the present invention is thus that further improved adsorbents are provided for the abovementioned methods and apparatuses.

The object is achieved by a method for removing carbon dioxide and, if appropriate, water from breathing air in closed or partially closed systems comprising the step
contacting the breathing air with a porous metal-organic framework material, the framework material comprising at least one at least bidentate organic compound which is bound by coordination to at least one metal ion.

The object is further achieved by a closed or partially closed system which comprises at least one breathing apparatus and also a breathing mask, a breathing suit or other life support system, further comprising a porous metal-organic framework material, the framework material comprising at least one at least bidentate organic compound which is bound by coordination to at least one metal ion.

This is because it has been found that the use of porous metal-organic framework materials in closed or partially closed systems which comprise at least one breathing apparatus and in methods for removing carbon dioxide and, if appropriate, water, from breathing air are particularly efficient and, in addition, can be readily regenerated.

To carry out the inventive method for removing carbon dioxide and, if appropriate, water, particular use can be made of a closed or partially closed system which comprises at least one breathing apparatus and also a breathing mask, a breathing suit, or other life support systems.

Closed systems are, in particular, those which have no opening to the surroundings through which atmospheric oxygen is to be introduced or removed.

Partially closed systems are, in particular, those in which no atmospheric oxygen is to be taken up into the system through the surroundings.

Surroundings of the closed or partially closed system which come into consideration are in principle any surroundings which do not contain surrounding gas or have a surrounding gas, the breathing of which does not ensure the necessary life support or freedom from harm of a human or higher animal.

Surroundings which contain no surrounding gas are situated, for example, under water or in space.

A surrounding gas, the breathing of which does not ensure the necessary life support or freedom from harm of a human or higher animal is, for example, air whose oxygen fraction or partial pressure is too low for breathing and/or which has other harmful constituents.

The breathing mask can be, for example, a mask such as is used in diving, therefore a diving mask. However, likewise, it can be a respiratory protection mask, as can be used, for example, in the case of fire, in a chemical accident, during painting or handling hazardous chemical or biological material, in extreme mountain climbing or at a great height (for example in an aircraft). In addition, such systems can also comprise suits. In addition, it is possible that the life support system is a helmet. Typically, such as a helmet can also be integrated into a corresponding suit. Frequently, in this connection, full protective suits can be mentioned. Space suits may also be mentioned in this context. Likewise, it can also be systems for rooms or passages of buildings, for example protective rooms, or of vehicles, for example in submarines, aircraft, in tunnels, mineshafts or the like.

The closed or partially closed system can in addition have a filter in which the porous metal-organic framework material is present at least as part of an adsorber bed. Other adsorbents such as zeolites can likewise be present.

The filter can be exchangeable or be installed fixed in the system. The filters to be used are known from the prior art. These are typically constituents of the systems which are likewise known in the prior art.

Preferably, the inventive closed or partially closed system is used for removing carbon dioxide and, if appropriate, water, from breathing air. In this case it is advantageous that, in addition to CO₂, also the water present in the breathing air can be removed. However, this is not a precondition for functioning of the CO₂ adsorption.

The porous metal-organic framework material is, inter alia, therefore advantageous because ready regeneration is possible.

Therefore, the present invention further relates to a method for regenerating a porous metal-organic framework material from a closed or partially closed system as has been described above comprising the steps
if appropriate removing the metal-organic framework material; and
impinging the framework material with a gas.

The gas can be, for example, air, nitrogen, an inert gas or a mixture thereof. Suitable inert gases are, for example, helium or argon.

The regeneration can be performed, for example, by simply passing the gas through the metal-organic framework material. Preferably, however, the regeneration takes place under pressure-swing and/or temperature-swing adsorption.

Therefore, it is preferred when the inventive method for regeneration is carried out in such a manner that the impingement takes place with the change of at least one parameter selected from pressure and temperature.

The term “pressure”, in the context of the present invention, is to be taken to mean the total pressure and/or the carbon dioxide partial pressure.

The regeneration of the metal-organic framework material can be performed during the use of the inventive closed or partially closed system.

The porous metal-organic framework material to be used is known in the prior art. The suitability of porous metal-organic framework materials for storage of carbon dioxide has been described, for example, by A. R. Millward et al., J. Am. Chem. Soc. 127 (2005), 17998-17999.

The porous metal-organic framework material comprises at least one at least bidentate, organic compound which is bound by coordination to a metal ion. This metal-organic framework material (MOF) is described, for example, in U.S. Pat. No. 5,648,508, EP-A-0 790 253, M. O'Keeffe et al., J. Sol. State Chem., 152 (2000), pages 3 to 20, H. Li et al., Nature 402 (1999), page 276, M. Eddaoudi et al., Topics in Catalysis 9 (1999), pages 105 to 111, B. Chen et al., Science 291 (2001), pages 1021 to 1023 and DE-A-101 11 230.

The MOFs according to the present invention comprise pores, in particular micropores and/or mesopores. Micropores are defined as those having a diameter of 2 nm or less and mesopores are defined by a diameter in the range from 2 to 50 nm, in each case in accordance with the definition as reported in Pure & Applied Chem. 57 (1985), 603-619, in particular page 606. The presence of micropores and/or mesopores can be checked with the aid of sorption measurements, these measurements determining the uptake capacity of the metal-organic framework material for nitrogen at 77 Kelvin as specified in DIN 66131 and/or DIN 66134.

Preferably, the specific surface area, calculated according to the Langmuir model (DIN 66131, 66134) for an MOF in powder form is greater than 5 m² μg, more preferably above 10 m²/g, more preferably greater than 50 m²/g, further more preferably greater than 500 m²/g, further more preferably greater than 1000 m²/g, and particularly preferably greater than 1500 m²/g.

MOF shaped bodies can have a lower specific surface area; but preferably greater than 10 m²/g, more preferably greater than 50 M²/g, further more preferably greater than 500 m²/g.

The metal component in the framework material according to the present invention is preferably selected from the groups Ia, IIa, IIIa, IVa to VIIIa and Ib to VIb. Particular preference is given to Mg, Ca, Sr, Ba, So, Y, Ti, Zr, Hf, V, Nb, Ta, Cr, Mo, W, Mn, Re, Fe, Ro, Os, Co, Rh, Ir, Ni, Pd, Pt, Cu, Ag, Au, Zn, Cd, Hg, Al, Ga, In, TI, Si, Ge, Sn, Pb, As, Sb and Bi. More preference is given to Zn, Cu, Ni, Pd, Pt, Ru, Rh and Co. In particular preference is given to Zn, Al, Ni and Cu. With respect to the ions of these elements, those which may particularly be mentioned are Mg²⁺, Ca²⁺, Sr²⁺, Ba²⁺, Sc³⁺, Y³⁺, Ti⁴⁺, Zr⁴⁺, Hf⁴⁺, V⁴⁺, V³⁺, V²⁺, Nb³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Ru²⁺, Os³⁺, Os²⁺, Co³⁺, Co²⁺Rh²⁺, Rh⁺, Ir²⁺, Ni²⁺, Ni⁺, Pd²⁺, Pd⁺, Pt⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Tl³⁺, Si⁴⁺, Si²⁺, Ge⁴⁺, Ge²⁺, Sn⁴⁺, Sn²⁺, Pb⁴⁺, Pb², As⁵⁺, As³⁺, As⁺, Sb⁵⁺, Sb³⁺, Sb⁺, Bi⁵⁺, Bi³⁺ and Bi⁺.

The term “at least bidentate organic compound” designates an organic compound which comprises at least one functional group which is able to form, to a given metal ion, at least two, preferably two coordinate, bonds and/or to two or more, preferably two, metal atoms in each case one coordinate bond.

As functional groups via which said coordinate bonds can be developed, in particular the following functional groups may be mentioned by way of example: —CO₂H, —CS₂H, —NO₂, —B(OH)₂, —SO₃H, —Si(OH))₃, —Ge(OH)₃, —Sn(OH)₃, —Si(SH)₄, —Ge(SH)₄, —Sn(SH)₃, —PO₃H, —AsO₃H, —AsO₄H, —P(SH)₃, —As(SH)₃, —CH(RSH)₂, —C(RSH)₃, —CH(RNH₂)₂, —C(RNH₂)₃, —CH(ROH)₂, —C(ROH)₃, —CH(RGN)₂, —C(RCN)₃, in which R, for example, can preferably be an alkylene group having 1, 2, 3, 4 or 5 carbon atoms such as, for example, a methylene, ethylene, n-propylene, isopropylene, n-butylene, isobutylene, tert-butylene or n-pentylene group, or an aryl group comprising one or two aromatic nuclei, such as, for example, 2 C₆ rings which can, if appropriate, be condensed and independently of one another can be suitably substituted with at least in each case one substituent, and/or which, independently of one another, can each comprise at least one heteroatom, such as, for example, N, O and/or S. According to likewise preferred embodiments, functional groups may be mentioned in which the abovementioned radical R is not present. In this respect, inter alia, —CH(SH)₂, —C(SH)₃, —CH(NH₂)₂, —C(NH₂)₃, CH(OH)₂, —C(OH)₃, —CH(CN)₂ or —C(CN)₃ may be mentioned.

The at least two functional groups can in principle be bound to any suitable organic compound provided that it is ensured that the organic compound having these functional groups is capable of forming the coordinate bond and for producing the framework material.

Preferably, the organic compounds which comprise the at least two functional groups are derived from a saturated or unsaturated aliphatic compound or an aromatic compound or a compound which is both aliphatic and aromatic.

The aliphatic compound or the aliphatic part of the compound which is both aliphatic and aromatic can be linear and/or branched and/or cyclic, a plurality of cycles per compound also being possible. Further preferably, the aliphatic compound or the aliphatic part of the compound which is both aliphatic and aromatic comprises 1 to 15, further preferably 1 to 14, further preferably 1 to 13, further preferably 1 to 12, further preferably 1 to 11, and in particular preferably 1 to 10, carbon atoms, such as, for example, 1, 2, 3, 4, 5, 6, 7, 8, 9 or 10 carbon atoms. In particular preference is given in this case to, inter alia, methane, adamantane, acetylene, ethylene or butadiene.

The aromatic compound or the aromatic part of the compound which is both aromatic and aliphatic can have one or else a plurality of nuclei, such as, for example, two, three, four or five nuclei, the nuclei being able to be present separately from one another and/or at least two nuclei in condensed form. Particularly preferably, the aromatic compound or the aromatic part of the compound which is both aliphatic and aromatic has one, two or three nuclei, one or two nuclei being particularly preferred. Independently of one another, in addition, each nucleus of said compound can comprise at least one heteroatom, such as, for example, N, O, S, B, P, Si, Al, preferably N, O and/or S. Further preferably, the aromatic compound or the aromatic part of the compound which is both aromatic and aliphatic comprises one or two C₆ nuclei, the two either being present separately of one another or in condensed form. In particular, as aromatic compounds, mention may be made of benzene, naphthalene and/or biphenyl and/or bipyridyl and/or pyridyl.

Particularly preferably, the at least bidentate, organic compound is derived from a di-, tri-, or tetracarboxylic acid, or their sulfur analogs. Sulfur analogs are the functional groups —C(═O)SH and also their tautomers and C(═S)SH which can be used instead of one or more carboxylic acid groups.

The term “derive” in the context of the present invention means that the at least bidentate, organic compound in the framework material can be present in partly deprotonated or completely deprotonated form. In addition, the at least bidentate, organic compound can comprise further substituents such as, for example, —OH, —NH₂, —OCH₃, —NH(CH₃), —N(CH₃)₂, —CN and also halides.

For example, in the context of the present invention, mention may be made of dicarboxylic acids, such as

oxalic acid, succinic acid, tartaric acid, 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, hepta-decanedicarboxylic acid, acetylenedicarboxylic acid, 1,2-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidodicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, peryienedicarboxylic acid, Pluriol E 200 dicarboxylic acid, 3,6-dioxa-octanedicarboxylic acid, 3,5-cyclohexadiene-1,2-dicarboxylic acid, octadicarboxylic acid, pentane-3,3-carboxylic acid, 4,4′-diamino-1,1′-biphenyl-3,3′-dicarboxylic acid, 4,4′-diaminobiphenyl-3,3′-dicarboxylic acid, benzidine-3,3′-dicarboxylic acid, 1,4-bis(phenylamino)benzene-2,5-dicarboxylic acid, 1,1′-binaphthyl-5,5′-dicarboxylic acid, 7-chloro-8-methylquinoIine-2,3-dicarboxylic acid, 1-anilinoanthraquinone-2,4′-dicarboxylic acid, polytetrahydrofuran-250-dicarboxylic acid, 1,4-bis(carboxymethyl)piperazine-2,3-dicarboxylic acid, 7-chloroquinoline-3,8-dicarboxylic acid, 1-(4-carboxy)phenyl-3-(4-chloro)phenylpyrazoline-4,5-dicarboxylic acid, 1,4,5,6,7,7-hexachloro-5-norbornene-2,3-dicarboxylic acid, phenylindanedicarboxylic acid, 1,3-dibenzyl-2-oxoimidazolidine-4,5-dicarboxylic acid, 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cis-dicarboxylic acid, 2,2′-biquinoline-4,4′-dicarboxylic acid, pyridine-3,4-dicarboxylic acid, 3,6,9-trioxaundecanedicarboxylic acid, O-hydroxybenzophenonedicarboxylic acid, Pluriol E 300 dicarboxylic acid, Pluriol E 400 dicarboxylic acid, Pluriol E 600 dicarboxylic acid, pyrazole-3,4-dicarboxylic acid, 2,3-pyrazinedicarboxylic acid, 5,6-dimethyl-2,3-pyrazinedicarboxylic acid, 4,4′-diaminodiphenyl ether diimidodicarboxylic acid, 4,4′-diaminodiphenylmethane diimidodicarboxylic acid, 4,4′-diaminodiphenyl sulfone diimidodicarboxylic acid, 2,6-naphthalenedicarboxylic acid, 1,3-adamantanedicarboxylic acid, 1,8-naphthalenedicarboxylic acid, 2,3-naphthalenedicarboxylic acid, 8 methoxy-2,3-naphthalenedicarboxylic acid, 8-nitro-2,3-naphthalenecarboxylic acid, 8-sulfo-2,3-naphthalenedicarboxylic acid, anthracene-2,3-dicarboxylic acid, 2′,3′-diphenyl-p-terphenyl-4,4″-dicarboxylic acid, diphenyl ether-4,4′-dicarboxylic acid, imidazole-4,5-dicarboxylic acid, 4(1H)-oxothiochromene-2,8-dicarboxylic acid, 5-tert-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptadicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluororubine-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrole-3,4-dicarboxylic acid, 1-benzyl-1H-pyrrole-3,4-dicarboxylic acid, anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid, 1,14-tetradecanedicarboxylic acid, 5,6-dehydronorbornane-2,3-dicarboxylic acids, or 5-ethyl-2,3-pyridinedicarboxylic acid,
tricarboxylic acid, such as
2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid, 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid or aurintricarboxylic acid,
or tetracarboxylic acids, such as
perylo[1,12-BCD]thiophene-1,1-dioxide-3,4,9,10-tetracarboxylic acid, perylene-tetracarboxylic acids such as perylene-3,4,9,10-tetracarboxylic acid or perylene 1,12-sulfone-3,4,9,10-tetracarboxylic acid, butanetetracarboxylic acid such as 1,2,3,4-butanetetracarboxylic acid or meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid, 1,2,5,6-hexanetetracarboxylic acid, 1,2,7,8-octanetetracarboxylic acid, 1,4,5,8-naphthalenetetracarboxylic acid, 1,2,9,10-decanetetracarboxylic acid, benzophenonetetracarboxylic acid, 3,3′,4, 4′-benzophenonetetracarboxylic acid, tetrahydrofurantetracarboxylic acid or cyclopentanetetracarboxylic acids such as cyclopentane-1,2,3,4-tetracarboxylic acid.

Very particularly preferably, use is made of, if appropriate at least monosubstituted, mono-, di-, tri-, tetranuclear or higher nuclear aromatic di-, tri- or tetracarboxylic acids, with each of the nuclei being able to comprise at least one heteroatom, with two or more nuclei being able to comprise identical or different heteroatoms. For example, preference is given to mononuclear dicarboxylic acids, mononuclear tricarboxylic acids, mononuclear tetracarboxylic acids, dinuclear dicarboxylic acids, dinuclear tricarboxylic acids, dinuclear tetracarboxylic acids, trinuclear dicarboxylic acids, trinuclear tricarboxylic acids, trinuclear tetracarboxylic acids, tetranuclear dicarboxylic acids, tetranuclear tricarboxylic acids and/or tetranuclear tetracarboxylic acids. Suitable heteroatoms are, for example, N, O, S, B, P, Si, Al, preferred heteroatoms here are N, S, and/or O. A suitable substituent which may be mentioned in this respect, is, inter alia, —OH, a nitro group, an amino group or an alkyl or alkoxy group.

In particular preferably, as at least bidentate, organic compounds, use is made of acetylenedicarboxylic acid (ADC), benzenedicarboxylic acids, naphthalenedicarboxylic acids, biphenyldicarboxylic acids, such as, for example, 4,4′-biphenyldicarboxylic acid (BPDC), bipyridinedicarboxylic acids, such as, for example, 2,2′-bipyridinedicarboxylic acids, such as, for example, 2,2′-bipyridine-5,5′-dicarboxylic acid, benzenetricarboxylic acids, such as, for example, 1,2,3-benzenetricarboxylic acid or 1,3,5-benzenetricarboxylic acid (BTC), adamantane tetracarboxylic acid (ATC), adamantane dibenzoate (ADB), benzene tribenzoate (BTB), methane tetrabenzoate (MTB), adamantane tetrabenzoate or dihydroxyterephthalic acids, such as, for example, 2,5-dihydroxyterephthalic acid (DHBDC).

Very particularly preferably, use is made of, inter alia, isophthalic acid, terephthalic acid, 2,5-dihydroxyterephthalic acid, 1,2,3-benzenetricarboxylic acid, 1,3,5-benzenetricarboxylic acid or 2,2′-bipyridine-5,5′-dicarboxylic acid.

In addition to these at least bidentate, organic compounds, the MOF can also comprise one or more unidentate ligands.

Suitable solvents for producing the MOF are, inter alia, ethanol, dimethylformamide, toluene, methanol, chlorobenzene, diethylformamide, dimethyl sulfoxide, water, hydrogen peroxide, methylamine, sodium hydroxide solution, N-methylpolidone ether, acetonitrile, benzyl chloride, triethylamine, ethylene glycol and mixtures thereof. Further metal ions, at least bidentate, organic compounds, and solvents for the production of MOF are described, inter alia, in U.S. Pat. No. 5,648,508 or DE-A 101 11 230.

The pore size of the MOF can be controlled by selection of suitable ligands and/or the at least bidentate, organic compound. In general it is true that the larger the organic compound, the larger the pore size. Preferably, the pore size is from 0.2 nm to 30 nm, particularly preferably the pore size is in the range from 0.3 nm to 3 nm, based on the crystalline material.

In an MOF shaped body, however, larger pores also occur, the size distribution of which can vary. Preferably, however, more than 50% of the total pore volume, in particular more than 75%, is formed by pores having a pore diameter of up to 1000 nm. Preferably, however, a majority of the pore volume is formed by pores of two diameter ranges. It is therefore further preferred if more than 25% of the total pore volume, in particular more than 50% total pore volume, is formed by pores which are in a diameter range from 100 nm to 800 nm and if more than 15% of the total pore volume, in particular more than 25% of the total pore volume, is formed by pores which are in a diameter range of up to 10 nm. The pore distribution can be determined by means of mercury porosimetry.

Examples of MOFs are given below. In addition to the designation of the MOF, the metal and also the at least bidentate ligand, furthermore the solvent and also the cell parameters (angles α, β and γ, and also the distances A, B and C in Å) are reported. The latter were determined by X-ray diffraction.

| | Constituents | | | | | | | | |
|---|---|---|---|---|---|---|---|---|---|
| | molar ratio | | | | | | | | Space |
| MOF-n | M + L | Solvents | α | β | γ | a | b | c | group |
| MOF-0 | Zn(NO₃)₂•6H₂O | Ethanol | 90 | 90 | 120 | 16.711 | 16.711 | 14.189 | P6(3)/ |
| | H₃(BTC) | | | | | | | | Mcm |
| MOF-2 | Zn(NO₃)₂•6H₂O | DMF | 90 | 102.8 | 90 | 6.718 | 15.49 | 12.43 | P2(1)/n |
| | (0.246 mmol) | Toluene | | | | | | | |
| | H₂(BDC) | | | | | | | | |
| | 0.241 mmol) | | | | | | | | |
| MOF-3 | Zn(NO₃)₂•6H₂O | DMF | 99.72 | 111.11 | 108.4 | 9.726 | 9.911 | 10.45 | P-1 |
| | (1.89 mmol) | MeOH | | | | | | | |
| | H₂(BDC) | | | | | | | | |
| | (1.93 mmol) | | | | | | | | |
| MOF-4 | Zn(NO₃)₂•6H₂O | Ethanol | 90 | 90 | 90 | 14.728 | 14.728 | 14.728 | P2(1)3 |
| | (1.00 mmol) | | | | | | | | |
| | H₃(BTC) | | | | | | | | |
| | (0.5 mmol) | | | | | | | | |
| MOF-5 | Zn(NO₃)₂•6H₂O | DMF | 90 | 90 | 90 | 25.669 | 25.669 | 25.669 | Fm-3m |
| | (2.22 mmol) | Chloro- | | | | | | | |
| | H₂(BDC) | benzene | | | | | | | |
| | (2.17 mmol) | | | | | | | | |
| MOF-38 | Zn(NO₃)₂•6H₂O | DMF | 90 | 90 | 90 | 20.657 | 20.657 | 17.84 | I4cm |
| | (0.27 mmol) | Chloro- | | | | | | | |
| | H₃(BTC) | benzene | | | | | | | |
| | (0.15 mmol) | | | | | | | | |
| MOF-31 | Zn(NO₃)₂•6H₂O | Ethanol | 90 | 90 | 90 | 10.821 | 10.821 | 10.821 | Pn(−3)m |
| Zn(ADC)₂ | 0.4 mmol | | | | | | | | |
| | H₂(ADC) | | | | | | | | |
| | 0.8 mmol | | | | | | | | |
| MOF-12 | Zn(NO₃)₂•6H₂O | Ethanol | 90 | 90 | 90 | 15.745 | 16.907 | 18.167 | Pbca |
| Zn₂(ATC) | 0.3 mmol | | | | | | | | |
| | H₄(ATC) | | | | | | | | |
| | 0.15 mmol | | | | | | | | |
| MOF-20 | Zn(NO₃)₂•6H₂O | DMF | 90 | 92.13 | 90 | 8.13 | 16.444 | 12.807 | P2(1)/c |
| ZnNDC | 0.37 mmol | Chloro- | | | | | | | |
| | H₂NDC | benzene | | | | | | | |
| | 0.36 mmol | | | | | | | | |
| MOF-37 | Zn(NO₃)₂•6H₂O | DEF | 72.38 | 83.16 | 84.33 | 9.952 | 11.576 | 15.556 | P-1 |
| | 0.2 mmol | Chloro- | | | | | | | |
| | H₂NDC | benzene | | | | | | | |
| | 0.2 mmol | | | | | | | | |
| MOF-8 | Tb(NO₃)₃•5H₂O | DMSO | 90 | 115.7 | 90 | 19.83 | 9.822 | 19.183 | C2/c |
| Tb₂ (ADC) | 0.10 mmol | MeOH | | | | | | | |
| | H₂ADC | | | | | | | | |
| | 0.20 mmol | | | | | | | | |
| MOF-9 | Tb(NO₃)₃•5H₂O | DMSO | 90 | 102.09 | 90 | 27.056 | 16.795 | 28.139 | C2/c |
| Tb₂ (ADC) | 0.08 mmol | | | | | | | | |
| | H₂ADB | | | | | | | | |
| | 0.12 mmol | | | | | | | | |
| MOF-6 | Tb(NO₃)₃•5H₂O | DMF | 90 | 91.28 | 90 | 17.599 | 19.996 | 10.545 | P21/c |
| | 0.30 mmol | MeOH | | | | | | | |
| | H₂ (BDC) | | | | | | | | |
| | 0.30 mmol | | | | | | | | |
| MOF-7 | Tb(NO₃)₃•5H₂O | H₂O | 102.3 | 91.12 | 101.5 | 6.142 | 10.069 | 10.096 | P-1 |
| | 0.15 mmol | | | | | | | | |
| | H₂(BDC) | | | | | | | | |
| | 0.15 mmol | | | | | | | | |
| MOF-69A | Zn(NO₃)₂•6H₂O | DEF | 90 | 111.6 | 90 | 23.12 | 20.92 | 12 | C2/c |
| | 0.083 mmol | H₂O₂ | | | | | | | |
| | 4,4′-BPDC | MeNH₂ | | | | | | | |
| | 0.041 mmol | | | | | | | | |
| MOF-69B | Zn(NO₃)₂•6H₂O | DEF | 90 | 95.3 | 90 | 20.17 | 18.55 | 12.16 | C2/c |
| | 0.083 mmol | H₂O₂ | | | | | | | |
| | 2,6-NCD | MeNH₂ | | | | | | | |
| | 0.041 mmol | | | | | | | | |
| MOF-11 | Cu(NO₃)₂•2.5H₂O | H₂O | 90 | 93.86 | 90 | 12.987 | 11.22 | 11.336 | C2/c |
| Cu₂(ATC) | 0.47 mmol | | | | | | | | |
| | H₂ATC | | | | | | | | |
| | 0.22 mmol | | | | | | | | |
| MOF-11 | | | 90 | 90 | 90 | 8.4671 | 8.4671 | 14.44 | P42/ |
| Cu₂(ATC) | | | | | | | | | mmc |
| dehydr. | | | | | | | | | |
| MOF-14 | Cu(NO₃)₂•2.5H₂O | H₂O | 90 | 90 | 90 | 26.946 | 26.946 | 26.946 | Im-3 |
| Cu₃ (BTB) | 0.28 mmol | DMF | | | | | | | |
| | H₃BTB | EtOH | | | | | | | |
| | 0.052 mmol | | | | | | | | |
| MOF-32 | Cd(NO₃)₂•4H₂O | H₂O | 90 | 90 | 90 | 13.468 | 13.468 | 13.468 | P(−4)3m |
| Cd(ATC) | 0.24 mmol | NaOH | | | | | | | |
| | H₄ATC | | | | | | | | |
| | 0.10 mmol | | | | | | | | |
| MOF-33 | ZnCl₂ | H₂O | 90 | 90 | 90 | 19.561 | 15.255 | 23.404 | Imma |
| Zn₂ (ATB) | 0.15 mmol | DMF | | | | | | | |
| | H₄ATB | EtOH | | | | | | | |
| | 0.02 mmol | | | | | | | | |
| MOF-34 | Ni(NO₃)₂•6H₂O | H₂O | 90 | 90 | 90 | 10.066 | 11.163 | 19.201 | P2₁2₁2₁ |
| Ni(ATC) | 0.24 mmol | NaOH | | | | | | | |
| | H₄ATC | | | | | | | | |
| | 0.10 mmol | | | | | | | | |
| MOF-36 | Zn(NO₃)₂•4H₂O | H₂O | 90 | 90 | 90 | 15.745 | 16.907 | 18.167 | Pbca |
| Zn₂ (MTB) | 0.20 mmol | DMF | | | | | | | |
| | H₄MTB | | | | | | | | |
| | 0.04 mmol | | | | | | | | |
| MOF-39 | Zn(NO₃)₂ 4H₂O | H₂O | 90 | 90 | 90 | 17.158 | 21.591 | 25.308 | Pnma |
| Zn₃O(HBTB) | 0.27 mmol | DMF | | | | | | | |
| | H₃BTB | EtOH | | | | | | | |
| | 0.07 mmol | | | | | | | | |
| NO305 | FeCl₂•4H₂O | DMF | 90 | 90 | 120 | 8.2692 | 8.2692 | 63.566 | R-3c |
| | 5.03 mmol | | | | | | | | |
| | formic acid | | | | | | | | |
| | 86.90 mmol | | | | | | | | |
| NO306A | FeCl₂•4H₂O | DEF | 90 | 90 | 90 | 9.9364 | 18.374 | 18.374 | Pbcn |
| | 5.03 mmol | | | | | | | | |
| | formic acid | | | | | | | | |
| | 86.90 mmol | | | | | | | | |
| NO29 | Mn(Ac)₂•4H₂O | DMF | 120 | 90 | 90 | 14.16 | 33.521 | 33.521 | P-1 |
| MOF-0 | 0.46 mmol | | | | | | | | |
| similar | H₃BTC | | | | | | | | |
| | 0.69 mmol | | | | | | | | |
| BPR48 | Zn(NO₃)₂ 6H₂O | DMSO | 90 | 90 | 90 | 14.5 | 17.04 | 18.02 | Pbca |
| A2 | 0.012 mmol | Toluene | | | | | | | |
| | H₂BDC | | | | | | | | |
| | 0.012 mmol | | | | | | | | |
| BPR69 | Cd(NO₃)₂ 4H₂O | DMSO | 90 | 98.76 | 90 | 14.16 | 15.72 | 17.66 | Cc |
| B1 | 0.0212 mmol | | | | | | | | |
| | H₂BDC | | | | | | | | |
| | 0.0428 mmol | | | | | | | | |
| BPR92 | Co(NO₃)₂•6H₂O | NMP | 106.3 | 107.63 | 107.2 | 7.5308 | 10.942 | 11.025 | P1 |
| A2 | 0.018 mmol | | | | | | | | |
| | H₂BDC | | | | | | | | |
| | 0.018 mmol | | | | | | | | |
| BPR95 | Cd(NO₃)₂ 4H₂O | NMP | 90 | 112.8 | 90 | 14.460 | 11.085 | 15.829 | P2(1)/n |
| C5 | 0.012 mmol | | | | | | | | |
| | H₂BDC | | | | | | | | |
| | 0.36 mmol | | | | | | | | |
| Cu C₆H₄O₆ | Cu(NO₃)₂•2.5H₂O | DMF | 90 | 105.29 | 90 | 15.259 | 14.816 | 14.13 | P2(1)/c |
| | 0.370 mmol | Chloro- | | | | | | | |
| | H₂BDC(OH)₂ | benzene | | | | | | | |
| | 0.37 mmol | | | | | | | | |
| M(BTC) | Co(SO₄) H₂O | DMF | as MOF-0 | | | | | | |
| MOF-0 | 0.055 mmol | | | | | | | | |
| similar | H₃BTC | | | | | | | | |
| | 0.037 mmol | | | | | | | | |
| Tb(C₆H₄O₆) | Tb(NO₃)₃•5H₂O | DMF | 104.6 | 107.9 | 97.147 | 10.491 | 10.981 | 12.541 | P-1 |
| | 0.370 mmol | Chloro- | | | | | | | |
| | H₂(C₆H₄O₆) | benzene | | | | | | | |
| | 0.56 mmol | | | | | | | | |
| Zn (C₂O₄) | ZnCl₂ | DMF | 90 | 120 | 90 | 9.4168 | 9.4168 | 8.464 | P(−3)1m |
| | 0.370 mmol | Chloro- | | | | | | | |
| | oxalic acid | benzene | | | | | | | |
| | 0.37 mmol | | | | | | | | |
| Co(CHO) | Co(NO₃)₂•5H₂O | DMF | 90 | 91.32 | 90 | 11.328 | 10.049 | 14.854 | P2(1)/n |
| | 0.043 mmol | | | | | | | | |
| | formic acid | | | | | | | | |
| | 1.60 mmol | | | | | | | | |
| Cd(CHO) | Cd(NO₃)₂•4H₂O | DMF | 90 | 120 | 90 | 8.5168 | 8.5168 | 22.674 | R-3c |
| | 0.185 mmol | | | | | | | | |
| | formic acid | | | | | | | | |
| | 0.185 mmol | | | | | | | | |
| Cu(C₃H₂O₄) | Cu(NO₃)₂•2.5H₂O | DMF | 90 | 90 | 90 | 8.366 | 8.366 | 11.919 | P43 |
| | 0.043 mmol | | | | | | | | |
| | malonic acid | | | | | | | | |
| | 0.192 mmol | | | | | | | | |
| Zn₆ (NDC)₅ | Zn(NO₃)₂•6H₂O | DMF | 90 | 95.902 | 90 | 19.504 | 16.482 | 14.64 | C2/m |
| MOF-48 | 0.097 mmol | Chloro- | | | | | | | |
| | 14 NDC | benzene | | | | | | | |
| | 0.069 mmol | H₂O₂ | | | | | | | |
| MOF-47 | Zn(NO₃)₂ 6H₂O | DMF | 90 | 92.55 | 90 | 11.303 | 16.029 | 17.535 | P2(1)/c |
| | 0.185 mmol | Chloro- | | | | | | | |
| | H₂(BDC[CH₃]₄) | benzene | | | | | | | |
| | 0.185 mmol | H₂O₂ | | | | | | | |
| MO25 | Cu(NO₃)₂•2.5H₂O | DMF | 90 | 112.0 | 90 | 23.880 | 16.834 | 18.389 | P2(1)/c |
| | 0.084 mmol | | | | | | | | |
| | BPhDC | | | | | | | | |
| | 0.085 mmol | | | | | | | | |
| Cu-Thio | Cu(NO₃)₂•2.5H₂O | DEF | 90 | 113.6 | 90 | 15.4747 | 14.514 | 14.032 | P2(1)/c |
| | 0.084 mmol | | | | | | | | |
| | thiophene | | | | | | | | |
| | dicarboxylic acid | | | | | | | | |
| | 0.085 mmol | | | | | | | | |
| ClBDC1 | Cu(NO₃)₂•2.5H₂O | DMF | 90 | 105.6 | 90 | 14.911 | 15.622 | 18.413 | C2/c |
| | 0.084 mmol | | | | | | | | |
| | H₂(BDCCl₂) | | | | | | | | |
| | 0.085 mmol | | | | | | | | |
| MOF-101 | Cu(NO₃)₂•2.5H₂O | DMF | 90 | 90 | 90 | 21.607 | 20.607 | 20.073 | Fm3m |
| | 0.084 mmol | | | | | | | | |
| | BrBDC | | | | | | | | |
| | 0.085 mmol | | | | | | | | |
| Zn₃(BTC)₂ | ZnCl₂ | DMF | 90 | 90 | 90 | 26.572 | 26.572 | 26.572 | Fm-3m |
| | 0.033 mmol | EtOH | | | | | | | |
| | H₃BTC | base | | | | | | | |
| | 0.033 mmol | added | | | | | | | |
| MOF-j | Co(CH₃CO₂)₂•4H₂O | H₂O | 90 | 112.0 | 90 | 17.482 | 12.963 | 6.559 | C2 |
| | (1.65 mmol) | | | | | | | | |
| | H₃(BZC) | | | | | | | | |
| | (0.95 mmol) | | | | | | | | |
| MOF-n | Zn(NO₃)₂•6H₂O | Ethanol | 90 | 90 | 120 | 16.711 | 16.711 | 14.189 | P6(3)/mcm |
| | H₃ (BTC) | | | | | | | | |
| PbBDC | Pb(NO₃)₂ | DMF | 90 | 102.7 | 90 | 8.3639 | 17.991 | 9.9617 | P2(1)/n |
| | (0.181 mmol) | Ethanol | | | | | | | |
| | H₂(BDC) | | | | | | | | |
| | (0.181 mmol) | | | | | | | | |
| Znhex | Zn(NO₃)₂•6H₂O | DMF | 90 | 90 | 120 | 37.1165 | 37.117 | 30.019 | P3(1)c |
| | (0.171 mmol) | p-Xylene | | | | | | | |
| | H₃BTB | Ethanol | | | | | | | |
| | (0.114 mmol) | | | | | | | | |
| AS16 | FeBr₂ | DMF | 90 | 90.13 | 90 | 7.2595 | 8.7894 | 19.484 | P2(1)c |
| | 0.927 mmol | anhydr. | | | | | | | |
| | H₂(BDC) | | | | | | | | |
| | 0.927 mmol | | | | | | | | |
| AS27-2 | FeBr₂ | DMF | 90 | 90 | 90 | 26.735 | 26.735 | 26.735 | Fm3m |
| | 0.927 mmol | anhydr. | | | | | | | |
| | H₃(BDC) | | | | | | | | |
| | 0.464 mmol | | | | | | | | |
| AS32 | FeCl₃ | DMF | 90 | 90 | 120 | 12.535 | 12.535 | 18.479 | P6(2)c |
| | 1.23 mmol | anhydr. | | | | | | | |
| | H₂(BDC) | Ethanol | | | | | | | |
| | 1.23 mmol | | | | | | | | |
| AS54-3 | FeBr₂ | DMF | 90 | 109.98 | 90 | 12.019 | 15.286 | 14.399 | C2 |
| | 0.927 | anhydr. | | | | | | | |
| | BPDC | n-Propanol | | | | | | | |
| | 0.927 mmol | | | | | | | | |
| AS61-4 | FeBr₂ | Pyridine | 90 | 90 | 120 | 13.017 | 13.017 | 14.896 | P6(2)c |
| | 0.927 mmol | anhydr. | | | | | | | |
| | m-BDC | | | | | | | | |
| | 0.927 mmol | | | | | | | | |
| AS68-7 | FeBr₂ | DMF | 90 | 90 | 90 | 18.3407 | 10.036 | 18.039 | Pca2₁ |
| | 0.927 mmol | anhydr. | | | | | | | |
| | m-BDC | Pyridine | | | | | | | |
| | 1.204 mmol | | | | | | | | |
| Zn(ADC) | Zn(NO₃)₂•6H₂O | DMF | 90 | 99.85 | 90 | 16.764 | 9.349 | 9.635 | C2/c |
| | 0.37 mmol | Chloro- | | | | | | | |
| | H₂(ADC) | benzene | | | | | | | |
| | 0.36 mmol | | | | | | | | |
| MOF-12 | Zn(NO₃)₂•6H₂O | Ethanol | 90 | 90 | 90 | 15.745 | 16.907 | 18.167 | Pbca |
| Zn₂ (ATC) | 0.30 mmol | | | | | | | | |
| | H₄(ATC) | | | | | | | | |
| | 0.15 mmol | | | | | | | | |
| MOF-20 | Zn(NO₃)₂•6H₂O | DMF | 90 | 92.13 | 90 | 8.13 | 16.444 | 12.807 | P2(1)/c |
| ZnNDC | 0.37 mmol | Chloro- | | | | | | | |
| | H₂NDC | benzene | | | | | | | |
| | 0.36 mmol | | | | | | | | |
| MOF-37 | Zn(NO₃)₂•6H₂O | DEF | 72.38 | 83.16 | 84.33 | 9.952 | 11.576 | 15.556 | P-1 |
| | 0.20 mmol | Chloro- | | | | | | | |
| | H₂NDC | benzene | | | | | | | |
| | 0.20 mmol | | | | | | | | |
| Zn(NDC) | Zn(NO₃)₂•6H₂O | DMSO | 68.08 | 75.33 | 88.31 | 8.631 | 10.207 | 13.114 | P-1 |
| (DMSO) | H₂NDC | | | | | | | | |
| Zn(NDC) | Zn(NO₃)₂•6H₂O | | 90 | 99.2 | 90 | 19.289 | 17.628 | 15.052 | C2/c |
| | H₂NDC | | | | | | | | |
| Zn(HPDC) | Zn(NO₃)₂•4H₂O | DMF | 107.9 | 105.06 | 94.4 | 8.326 | 12.085 | 13.767 | P-1 |
| | 0.23 mmol | H₂O | | | | | | | |
| | H₂(HPDC) | | | | | | | | |
| | 0.05 mmol | | | | | | | | |
| Co(HPDC) | Co(NO₃)₂•6H₂O | DMF | 90 | 97.69 | 90 | 29.677 | 9.63 | 7.981 | C2/c |
| | 0.21 mmol | H₂O/ | | | | | | | |
| | H₂ (HPDC) | Ethanol | | | | | | | |
| | 0.06 mmol | | | | | | | | |
| Zn₃(PDC)2.5 | Zn(NO₃)₂•4H₂O | DMF/ | 79.34 | 80.8 | 85.83 | 8.564 | 14.046 | 26.428 | P-1 |
| | 0.17 mmol | ClBz | | | | | | | |
| | H₂(HPDC) | H₂0/ | | | | | | | |
| | 0.05 mmol | TEA | | | | | | | |
| Cd₂ | Cd(NO₃)₂•4H₂O | Methanol/ | 70.59 | 72.75 | 87.14 | 10.102 | 14.412 | 14.964 | P-1 |
| (TPDC)2 | 0.06 mmol | CHP | | | | | | | |
| | H₂(HPDC) | H₂O | | | | | | | |
| | 0.06 mmol | | | | | | | | |
| Tb(PDC)1.5 | Tb(NO₃)₃•5H₂O | DMF | 109.8 | 103.61 | 100.14 | 9.829 | 12.11 | 14.628 | P-1 |
| | 0.21 mmol | H₂O/ | | | | | | | |
| | H₂(PDC) | Ethanol | | | | | | | |
| | 0.034 mmol | | | | | | | | |
| ZnDBP | Zn(NO₃)₂•6H₂O | MeOH | 90 | 93.67 | 90 | 9.254 | 10.762 | 27.93 | P2/n |
| | 0.05 mmol | | | | | | | | |
| | dibenzyl phosphate | | | | | | | | |
| | 0.10 mmol | | | | | | | | |
| Zn₃(BPDC) | ZnBr₂ | DMF | 90 | 102.76 | 90 | 11.49 | 14.79 | 19.18 | P21/n |
| | 0.021 mmol | | | | | | | | |
| | 4,4′BPDC | | | | | | | | |
| | 0.005 mmol | | | | | | | | |
| CdBDC | Cd(NO₃)₂•4H₂O | DMF | 90 | 95.85 | 90 | 11.2 | 11.11 | 16.71 | P21/n |
| | 0.100 mmol | Na₂SiO₃ | | | | | | | |
| | H₂(BDC) | (aq) | | | | | | | |
| | 0.401 mmol | | | | | | | | |
| Cd-mBDC | Cd(NO₃)₂•4H₂O | DMF | 90 | 101.1 | 90 | 13.69 | 18.25 | 14.91 | C2/c |
| | 0.009 mmol | MeNH₂ | | | | | | | |
| | H₂(mBDC) | | | | | | | | |
| | 0.018 mmol | | | | | | | | |
| Zn₄OBNDC | Zn(NO₃)₂•6H₂O | DEF | 90 | 90 | 90 | 22.35 | 26.05 | 59.56 | Fmmm |
| | 0.041 mmol | MeNH₂ | | | | | | | |
| | BNDC | H₂O₂ | | | | | | | |
| Eu(TCA) | Eu(NO₃)₃•6H₂O | DMF | 90 | 90 | 90 | 23.325 | 23.325 | 23.325 | Pm-3n |
| | 0.14 mmol | Chloro- | | | | | | | |
| | TCA | benzene | | | | | | | |
| | 0.026 mmol | | | | | | | | |
| Tb(TCA) | Tb(NO₃)₃•6H₂O | DMF | 90 | 90 | 90 | 23.272 | 23.272 | 23.372 | Pm-3n |
| | 0.069 mmol | Chloro- | | | | | | | |
| | TCA | benzene | | | | | | | |
| | 0.026 mmol | | | | | | | | |
| Formates | Ce(NO₃)₃•6H₂O | H₂O | 90 | 90 | 120 | 10.668 | 10.667 | 4.107 | R-3m |
| | 0.138 mmol | Ethanol | | | | | | | |
| | formic acid | | | | | | | | |
| | 0.43 mmol | | | | | | | | |
| | FeCl₂•4H₂O | DMF | 90 | 90 | 120 | 8.2692 | 8.2692 | 63.566 | R-3c |
| | 5.03 mmol | | | | | | | | |
| | formic acid | | | | | | | | |
| | 86.90 mmol | | | | | | | | |
| | FeCl₂•4H₂O | DEF | 90 | 90 | 90 | 9.9364 | 18.374 | 18.374 | Pbcn |
| | 5.03 mmol | | | | | | | | |
| | formic acid | | | | | | | | |
| | 86.90 mmol | | | | | | | | |
| | FeCl₂•4H₂O | DEF | 90 | 90 | 90 | 8.335 | 8.335 | 13.34 | P-31c |
| | 5.03 mmol | | | | | | | | |
| | formic acid | | | | | | | | |
| | 86.90 mmol | | | | | | | | |
| NO330 | FeCl₂•4H₂O | Formamide | 90 | 90 | 90 | 8.7749 | 11.655 | 8.3297 | Pnna |
| | 0.50 mmol | | | | | | | | |
| | formic acid | | | | | | | | |
| | 8.69 mmol | | | | | | | | |
| NO332 | FeCl₂•4H₂O | DIP | 90 | 90 | 90 | 10.0313 | 18.808 | 18.355 | Pbcn |
| | 0.50 mmol | | | | | | | | |
| | formic acid | | | | | | | | |
| | 8.69 mmol | | | | | | | | |
| NO333 | FeCl₂•4H₂O | DBF | 90 | 90 | 90 | 45.2754 | 23.861 | 12.441 | Cmcm |
| | 0.50 mmol | | | | | | | | |
| | formic acid | | | | | | | | |
| | 8.69 mmol | | | | | | | | |
| NO335 | FeCl₂•4H₂O | CHF | 90 | 91.372 | 90 | 11.5964 | 10.187 | 14.945 | P21/n |
| | 0.50 mmol | | | | | | | | |
| | formic acid | | | | | | | | |
| | 8.69 mmol | | | | | | | | |
| NO336 | FeCl₂•4H₂O | MFA | 90 | 90 | 90 | 11.7945 | 48.843 | 8.4136 | Pbcm |
| | 0.50 mmol | | | | | | | | |
| | formic acid | | | | | | | | |
| | 8.69 mmol | | | | | | | | |
| NO13 | Mn(Ac)₂•4H₂O | Ethanol | 90 | 90 | 90 | 18.66 | 11.762 | 9.418 | Pbcn |
| | 0.46 mmol | | | | | | | | |
| | benzoic acid | | | | | | | | |
| | 0.92 mmol | | | | | | | | |
| | bipyridine | | | | | | | | |
| | 0.46 mmol | | | | | | | | |
| NO29 | Mn(Ac)₂•4H₂O | DMF | 120 | 90 | 90 | 14.16 | 33.521 | 33.521 | P-1 |
| MOF-0 | 0.46 mmol | | | | | | | | |
| similar | H₃BTC | | | | | | | | |
| | 0.69 mmol | | | | | | | | |
| Mn(hfac)₂ | Mn(Ac)₂•4H₂O | Ether | 90 | 95.32 | 90 | 9.572 | 17.162 | 14.041 | C2/c |
| (O₂CC₆H₅) | 0.46 mmol | | | | | | | | |
| | Hfac | | | | | | | | |
| | 0.92 mmol | | | | | | | | |
| | bipyridine | | | | | | | | |
| | 0.46 mmol | | | | | | | | |
| BPR43G2 | Zn(NO₃)₂•6H₂O | DMF | 90 | 91.37 | 90 | 17.96 | 6.38 | 7.19 | C2/c |
| | 0.0288 mmol | CH₃CN | | | | | | | |
| | H₂BDC | | | | | | | | |
| | 0.0072 mmol | | | | | | | | |
| BPR48A2 | Zn(NO₃)₂ 6H₂O | DMSO | 90 | 90 | 90 | 14.5 | 17.04 | 18.02 | Pbca |
| | 0.012 mmol | Toluene | | | | | | | |
| | H₂BDC | | | | | | | | |
| | 0.012 mmol | | | | | | | | |
| BPR49B1 | Zn(NO₃)₂ 6H₂O | DMSO | 90 | 91.172 | 90 | 33.181 | 9.824 | 17.884 | C2/c |
| | 0.024 mmol | Methanol | | | | | | | |
| | H₂BDC | | | | | | | | |
| | 0.048 mmol | | | | | | | | |
| BPR56E1 | Zn(NO₃)₂ 6H₂O | DMSO | 90 | 90.096 | 90 | 14.5873 | 14.153 | 17.183 | P2(1)/n |
| | 0.012 mmol | n- | | | | | | | |
| | H₂BDC | Propanol | | | | | | | |
| | 0.024 mmol | | | | | | | | |
| BPR68D10 | Zn(NO₃)₂ 6H₂O | DMSO | 90 | 95.316 | 90 | 10.0627 | 10.17 | 16.413 | P2(1)/c |
| | 0.0016 mmol | Benzene | | | | | | | |
| | H₃BTC | | | | | | | | |
| | 0.0064 mmol | | | | | | | | |
| BPR69B1 | Cd(NO₃)₂ 4H₂O | DMSO | 90 | 98.76 | 90 | 14.16 | 15.72 | 17.66 | Cc |
| | 0.0212 mmol | | | | | | | | |
| | H₂BDC | | | | | | | | |
| | 0.0428 mmol | | | | | | | | |
| BPR73E4 | Cd(NO₃)₂ 4H₂O | DMSO | 90 | 92.324 | 90 | 8.7231 | 7.0568 | 18.438 | P2(1)/n |
| | 0.006 mmol | Toluene | | | | | | | |
| | H₂BDC | | | | | | | | |
| | 0.003 mmol | | | | | | | | |
| BPR76D5 | Zn(NO₃)₂ 6H₂O | DMSO | 90 | 104.17 | 90 | 14.4191 | 6.2599 | 7.0611 | Pc |
| | 0.0009 mmol | | | | | | | | |
| | H₂BzPDC | | | | | | | | |
| | 0.0036 mmol | | | | | | | | |
| BPR80B5 | Cd(NO₃)₂•4H₂O | DMF | 90 | 115.11 | 90 | 28.049 | 9.184 | 17.837 | C2/c |
| | 0.018 mmol | | | | | | | | |
| | H₂BDC | | | | | | | | |
| | 0.036 mmol | | | | | | | | |
| BPR80H5 | Cd(NO₃)₂ 4H₂O | DMF | 90 | 119.06 | 90 | 11.4746 | 6.2151 | 17.268 | P2/c |
| | 0.027 mmol | | | | | | | | |
| | H₂BDC | | | | | | | | |
| | 0.027 mmol | | | | | | | | |
| BPR82C6 | Cd(NO₃)₂ 4H₂O | DMF | 90 | 90 | 90 | 9.7721 | 21.142 | 27.77 | Fdd2 |
| | 0.0068 mmol | | | | | | | | |
| | H₂BDC | | | | | | | | |
| | 0.202 mmol | | | | | | | | |
| BPR86C3 | Co(NO₃)₂ 6H₂O | DMF | 90 | 90 | 90 | 18.3449 | 10.031 | 17.983 | Pca2(1) |
| | 0.0025 mmol | | | | | | | | |
| | H₂BDC | | | | | | | | |
| | 0.075 mmol | | | | | | | | |
| BPR86H6 | Cd(NO₃)₂•6H₂O | DMF | 80.98 | 89.69 | 83.412 | 9.8752 | 10.263 | 15.362 | P-1 |
| | 0.010 mmol | | | | | | | | |
| | H₂BDC | | | | | | | | |
| | 0.010 mmol | | | | | | | | |
| | Co(NO₃)₂ 6H₂O | NMP | 106.3 | 107.63 | 107.2 | 7.5308 | 10.942 | 11.025 | P1 |
| BPR95A2 | Zn(NO₃)₂ 6H₂O | NMP | 90 | 102.9 | 90 | 7.4502 | 13.767 | 12.713 | P2(1)/c |
| | 0.012 mmol | | | | | | | | |
| | H₂BDC | | | | | | | | |
| | 0.012 mmol | | | | | | | | |
| CuC₆F₄O₄ | Cu(NO₃)₂•2.5H₂O | DMF | 90 | 98.834 | 90 | 10.9675 | 24.43 | 22.553 | P2(1)/n |
| | 0.370 mmol | Chloro- | | | | | | | |
| | H₂BDC(OH)₂ | benzene | | | | | | | |
| | 0.37 mmol | | | | | | | | |
| Fe Formic | FeCl₂•4H₂O | DMF | 90 | 91.543 | 90 | 11.495 | 9.963 | 14.48 | P2(1)/n |
| | 0.370 mmol | | | | | | | | |
| | formic acid | | | | | | | | |
| | 0.37 mmol | | | | | | | | |
| Mg Formic | Mg(NO₃)₂•6H₂O | DMF | 90 | 91.359 | 90 | 11.383 | 9.932 | 14.656 | P2(1)/n |
| | 0.370 mmol | | | | | | | | |
| | formic acid | | | | | | | | |
| | 0.37 mmol | | | | | | | | |
| MgC₆H₄O₆ | Mg(NO₃)₂•6H₂O | DMF | 90 | 96.624 | 90 | 17.245 | 9.943 | 9.273 | C2/c |
| | 0.370 mmol | | | | | | | | |
| | H₂BDC(OH)₂ | | | | | | | | |
| | 0.37 mmol | | | | | | | | |
| Zn C₂H₄BDC | ZnCl₂ | DMF | 90 | 94.714 | 90 | 7.3386 | 16.834 | 12.52 | P2(1)/n |
| MOF-38 | 0.44 mmol | | | | | | | | |
| | CBBDC | | | | | | | | |
| | 0.261 mmol | | | | | | | | |
| MOF-49 | ZnCl₂ | DMF | 90 | 93.459 | 90 | 13.509 | 11.984 | 27.039 | P2/c |
| | 0.44 mmol | CH₃CN | | | | | | | |
| | m-BDC | | | | | | | | |
| | 0.261 mmol | | | | | | | | |
| MOF-26 | Cu(NO₃)₂•5H₂O | DMF | 90 | 95.607 | 90 | 20.8797 | 16.017 | 26.176 | P2(1)/n |
| | 0.084 mmol | | | | | | | | |
| | DCPE | | | | | | | | |
| | 0.085 mmol | | | | | | | | |
| MOF-112 | Cu(NO₃)₂•2.5H₂O | DMF | 90 | 107.49 | 90 | 29.3241 | 21.297 | 18.069 | C2/c |
| | 0.084 mmol | Ethanol | | | | | | | |
| | o-Br-m-BDC | | | | | | | | |
| | 0.085 mmol | | | | | | | | |
| MOF-109 | Cu(NO₃)₂•2.5H₂O | DMF | 90 | 111.98 | 90 | 23.8801 | 16.834 | 18.389 | P2(1)/c |
| | 0.084 mmol | | | | | | | | |
| | KDB | | | | | | | | |
| | 0.085 mmol | | | | | | | | |
| MOF-111 | Cu(NO₃)₂•2.5H₂O | DMF | 90 | 102.16 | 90 | 10.6767 | 18.781 | 21.052 | C2/c |
| | 0.084 mmol | Ethanol | | | | | | | |
| | o-BrBDC | | | | | | | | |
| | 0.085 mmol | | | | | | | | |
| MOF-110 | Cu(NO₃)₂•2.5H₂O | DMF | 90 | 90 | 120 | 20.0652 | 20.065 | 20.747 | R-3/m |
| | 0.084 mmol | | | | | | | | |
| | thiophene | | | | | | | | |
| | dicarboxylic acid | | | | | | | | |
| | 0.085 mmol | | | | | | | | |
| MOF-107 | Cu(NO₃)₂•2.5H₂O | DEF | 104.8 | 97.075 | 95.206 | 11.032 | 18.067 | 18.452 | P-1 |
| | 0.084 mmol | | | | | | | | |
| | thiophene | | | | | | | | |
| | dicarboxylic acid | | | | | | | | |
| | 0.085 mmol | | | | | | | | |
| MOF-108 | Cu(NO₃)₂•2.5H₂O | DBF/ | 90 | 113.63 | 90 | 15.4747 | 14.514 | 14.032 | C2/c |
| | 0.084 mmol | Methanol | | | | | | | |
| | thiophene | | | | | | | | |
| | dicarboxylic acid | | | | | | | | |
| | 0.085 mmol | | | | | | | | |
| MOF-102 | Cu(NO₃)₂•2.5H₂O | DMF | 91.63 | 106.24 | 112.01 | 9.3845 | 10.794 | 10.831 | P-1 |
| | 0.084 mmol | | | | | | | | |
| | H₂(BDCCl₂) | | | | | | | | |
| | 0.085 mmol | | | | | | | | |
| Clbdc1 | Cu(NO₃)₂•2.5H₂O | DEF | 90 | 105.56 | 90 | 14.911 | 15.622 | 18.413 | P-1 |
| | 0.084 mmol | | | | | | | | |
| | H₂(BDCCl₂) | | | | | | | | |
| | 0.085 mmol | | | | | | | | |
| Cu(NMOP) | Cu(NO₃)₂•2.5H₂O | DMF | 90 | 102.37 | 90 | 14.9238 | 18.727 | 15.529 | P2(1)/m |
| | 0.084 mmol | | | | | | | | |
| | NBDC | | | | | | | | |
| | 0.085 mmol | | | | | | | | |
| Tb(BTC) | Tb(NO₃)₃•5H₂O | DMF | 90 | 106.02 | 90 | 18.6986 | 11.368 | 19.721 | |
| | 0.033 mmol | | | | | | | | |
| | H₃BTC | | | | | | | | |
| | 0.033 mmol | | | | | | | | |
| Zn₃(BTC)₂ | ZnCl₂ | DMF | 90 | 90 | 90 | 26.572 | 26.572 | 26.572 | Fm-3m |
| | 0.033 mmol | Ethanol | | | | | | | |
| | H₃BTC | | | | | | | | |
| | 0.033 mmol | | | | | | | | |
| Zn₄O(NDC) | Zn(NO₃)₂•4H₂O | DMF | 90 | 90 | 90 | 41.5594 | 18.818 | 17.574 | aba2 |
| | 0.066 mmol | Ethanol | | | | | | | |
| | 14NDC | | | | | | | | |
| | 0.066 mmol | | | | | | | | |
| CdTDC | Cd(NO₃)₂•4H₂O | DMF | 90 | 90 | 90 | 12.173 | 10.485 | 7.33 | Pmma |
| | 0.014 mmol | H₂O | | | | | | | |
| | thiophene | | | | | | | | |
| | 0.040 mmol | | | | | | | | |
| | DABCO | | | | | | | | |
| | 0.020 mmol | | | | | | | | |
| IRMOF-2 | Zn(NO₃)₂•4H₂O | DEF | 90 | 90 | 90 | 25.772 | 25.772 | 25.772 | Fm-3m |
| | 0.160 mmol | | | | | | | | |
| | o-Br-BDC | | | | | | | | |
| | 0.60 mmol | | | | | | | | |
| IRMOF-3 | Zn(NO₃)₂•4H₂O | DEF | 90 | 90 | 90 | 25.747 | 25.747 | 25.747 | Fm-3m |
| | 0.20 mmol | Ethanol | | | | | | | |
| | H₂N-BDC | | | | | | | | |
| | 0.60 mmol | | | | | | | | |
| IRMOF-4 | Zn(NO₃)₂•4H₂O | DEF | 90 | 90 | 90 | 25.849 | 25.849 | 25.849 | Fm-3m |
| | 0.11 mmol | | | | | | | | |
| | [C₃H₇O]₂-BDC | | | | | | | | |
| | 0.48 mmol | | | | | | | | |
| IRMOF-5 | Zn(NO₃)₂•4H₂O | DEF | 90 | 90 | 90 | 12.882 | 12.882 | 12.882 | Pm-3m |
| | 0.13 mmol | | | | | | | | |
| | [C₅H₁₁O]₂-BDC | | | | | | | | |
| | 0.50 mmol | | | | | | | | |
| IRMOF-6 | Zn(NO₃)₂•4H₂O | DEF | 90 | 90 | 90 | 25.842 | 25.842 | 25.842 | Fm-3m |
| | 0.20 mmol | | | | | | | | |
| | [C₂H₄]-BDC | | | | | | | | |
| | 0.60 mmol | | | | | | | | |
| IRMOF-7 | Zn(NO₃)₂•4H₂O | DEF | 90 | 90 | 90 | 12.914 | 12.914 | 12.914 | Pm-3m |
| | 0.07 mmol | | | | | | | | |
| | 1,4NDC | | | | | | | | |
| | 0.20 mmol | | | | | | | | |
| IRMOF-8 | Zn(NO₃)₂•4H₂O | DEF | 90 | 90 | 90 | 30.092 | 30.092 | 30.092 | Fm-3m |
| | 0.55 mmol | | | | | | | | |
| | 2,6NDC | | | | | | | | |
| | 0.42 mmol | | | | | | | | |
| IRMOF-9 | Zn(NO₃)₂•4H₂O | DEF | 90 | 90 | 90 | 17.147 | 23.322 | 25.255 | Pnnm |
| | 0.05 mmol | | | | | | | | |
| | BPDC | | | | | | | | |
| | 0.42 mmol | | | | | | | | |
| IRMOF-10 | Zn(NO₃)₂•4H₂O | DEF | 90 | 90 | 90 | 34.281 | 34.281 | 34.281 | Fm-3m |
| | 0.02 mmol | | | | | | | | |
| | BPDC | | | | | | | | |
| | 0.012 mmol | | | | | | | | |
| IRMOF-11 | Zn(NO₃)₂•4H₂O | DEF | 90 | 90 | 90 | 24.822 | 24.822 | 56.734 | R-3m |
| | 0.05 mmol | | | | | | | | |
| | HPDC | | | | | | | | |
| | 0.20 mmol | | | | | | | | |
| IRMOF-12 | Zn(NO₃)₂•4H₂O | DEF | 90 | 90 | 90 | 34.281 | 34.281 | 34.281 | Fm-3m |
| | 0.017 mmol | | | | | | | | |
| | HPDC | | | | | | | | |
| | 0.12 mmol | | | | | | | | |
| IRMOF-13 | Zn(NO₃)₂•4H₂O | DEF | 90 | 90 | 90 | 24.822 | 24.822 | 56.734 | R-3m |
| | 0.048 mmol | | | | | | | | |
| | PDC | | | | | | | | |
| | 0.31 mmol | | | | | | | | |
| IRMOF-14 | Zn(NO₃)₂•4H₂O | DEF | 90 | 90 | 90 | 34.381 | 34.381 | 34.381 | Fm-3m |
| | 0.17 mmol | | | | | | | | |
| | PDC | | | | | | | | |
| | 0.12 mmol | | | | | | | | |
| IRMOF-15 | Zn(NO₃)₂•4H₂O | DEF | 90 | 90 | 90 | 21.459 | 21.459 | 21.459 | Im-3m |
| | 0.063 mmol | | | | | | | | |
| | TPDC | | | | | | | | |
| | 0.025 mmol | | | | | | | | |
| IRMOF-16 | Zn(NO₃)₂•4H₂O | DEF | 90 | 90 | 90 | 21.49 | 21.49 | 21.49 | Pm-3m |
| | 0.0126 mmol | NMP | | | | | | | |
| | TPDC | | | | | | | | |
| | 0.05 mmol | | | | | | | | |

ADC Acetylenedicarboxylic acid
NDC Naphthalenedicarboxylic acid
BDC Benzenedicarboxylic acid
ATC Adamantanetetracarboxylic acid
BTC Benzenetricarboxylic acid
BTB Benzenetribenzoic acid
MTB Methanetetrabenzoic acid
ATB Adamantanetetrabenzoic acid
ADB Adamantanedibenzoic acid

Further metal-organic framework materials are MOF-2 to 4, MOF-9, MOF-31 to 36, MOF-39, MOF-69 to 80, MOF103 to 106, MOF-122, MOF-125, MOF-150, MOF-177, MOF-178, MOF-235, MOF-236, MOF-500, MOF-501, MOF-502, MOF-505, IRMOF-1, IRMOF-61, IRMOP-13, IRMOP-51, MIL-17, MIL-45, MIL-47, MIL-53, MIL-59, MIL-60, MIL-61, MIL-63, MIL-68, MIL-79, MIL-80, MIL-83, MIL-85, CPL-1 to 2, SZL-1 which are described in the literature.

In particular preference is given to a porous metal-organic framework material in which Zn, Al or Cu is present as metal ion and the at least bidentate, organic compound is terephthalic acid, isophthalic acid, 2,6-naphthalenedicarboxylic acid or 1,3,5-benzenetricarboxylic acid.

In addition to the conventional method for production of MOFs, as described, for example, in U.S. Pat. No. 5,648,508, they can also be produced by the electrochemical route. In this respect, reference is made to DE-A 103 55 087 and also WO-A 2005/049892. The MOFs produced in this way exhibit particularly good properties in relation to adsorption and desorption of chemical substances, in particular gases. They thus differ from those which are produced conventionally, even when these are formed from the same organic and metal ion constituents and are therefore to be considered novel framework materials. In the context of the present invention, electrochemically produced MOFs are particularly preferred.

Consequently, the electrochemical production relates to a crystalline porous metal-organic framework material comprising at least one at least bidentate, organic compound which is bound by coordination to at least one metal ion and which is obtained in a reaction medium comprising the at least one bidentate organic compound by at least one metal ion being generated by oxidation of at least one anode comprising the corresponding metal.

The term “electrochemical production” designates a production method in which the formation of at least one reaction product is associated with the migration of electric charges or the occurrence of electric potentials.

The term “at least one metal ion”, as used in connection with the electrochemical production, designates embodiments according to which at least one ion of a metal or at least one ion of a first metal and at least one ion of at least one second metal different from the first metal are provided by anodic oxidation.

Consequently, the electrochemical production comprises embodiments in which at least one ion of at least one metal is provided by anodic oxidation and at least one ion of at least one metal is provided via a metal salt, the at least one metal in the metal salt and the at least one metal which is provided as metal ion via anodic oxidation can be identical or different from one another. Therefore the present invention, with respect to electrochemically produced MOFs, comprises, for example, an embodiment according to which the reaction medium comprises one or more different salts of a metal and the metal ion present in this salt or in these salts is additionally provided by anodic oxidation of at least one anode comprising this metal. Likewise, the reaction medium can comprise one or more different salts of at least one metal and at least one metal different from these metals can be provided by anodic oxidation as metal ion in the reaction medium.

According to a preferred embodiment of the present invention in connection with the electrochemical production, the at least one metal ion is provided by anodic oxidation of at least one anode comprising this at least one metal, though no further metal being provided via a metal salt.

The term “metal”, as used in the context of the present invention in connection with the electrochemical production of MOFS, comprises all elements of the Periodic Table of the Elements which can be provided via anodic oxidation via the electrochemical route in a reaction medium and together with at least one at least bidentate, organic compound are able to form at least one metal-organic porous framework material.

Independently of its production, the resultant MOF occurs in pulverulent form or as agglomerate. This can be used as such as sorbent in the inventive method alone or together with other sorbents or further materials. Preferably this occurs as bulk material, in particular in a fixed bed. In addition, the MOF can be converted into a shaped body. Preferred methods in this case are rod extrusion or tableting. In the shaped body production, further materials, such as, for example, binders, lubricants or other additives, can be added to the MOF. Likewise, it is conceivable that mixtures of MOF and other adsorbents, for example activated carbon, are produced as shaped bodies or separately result in shaped bodies which are then used as shaped body mixtures.

With respect to the possible geometries of these MOF shaped bodies, there are essentially no restrictions. For example, mention may be made of, inter alia, pellets, such as, for example, disk-shaped pellets, pills, spheres, granules, extrudates, for example rod extrudates, honeycombs, meshes or hollow bodies.

For the production of these shaped bodies, in principle all suitable methods are possible. In particular, the following procedures are preferred:
kneading the framework material alone or together with at least one binder and/or at least one pasting agent and/or at least one template compound to obtain a mixture; shaping the resultant mixture by means of at least one suitable method, such as, for example, extrusion; optionally washing and/or drying and/or calcining the extrudate; optionally final processing.
Applying the framework material to at least one, if appropriate, porous support material. The resultant material can then be further processed to give a shaped body in accordance with the above described method.
Applying the framework material to at least one, if appropriate, porous substrate.
Foaming into porous plastics, such as, e.g. polyurethane.

Kneading and shaping can proceed according to any suitable method, such as, for example, as described in Ullmanns Enzyklopadie der Technischen Chemie [Ulimann's Encyclopedia of Industrial Chemistry], 4th edition, volume 2, pp. 313 ff. (1972), the contents of which in this respect are hereby incorporated in entirety by reference into the context of the present application.

For example, preferably, the kneading and/or shaping can proceed by means of a piston press, roller press in the presence or absence of at least one binder, compounding, pelleting, tableting, extrusion, co-extrusion, foaming, spinning, coating, granulating, preferably spray-granulating, spraying, spray-drying or a combination of two or more of these methods.

Very particularly, pellets and/or tablets are produced.

The kneading and/or shaping can proceed at elevated temperatures, such as, for example, in the range from room temperature to 300° C. and/or at elevated pressure, such as, for example, in the range from atmospheric pressure up to a few 100 bar and/or in a protective gas atmosphere such as, for example, in the presence of at least one noble gas, nitrogen, or a mixture of two or more thereof.

The kneading and/or shaping is carried out according to a further embodiment with addition of at least one binder, as binder, use being able to be made in principle of any chemical compound which ensures the viscosity of the mix to be kneaded and/or shaped desired for kneading and/or shaping. Consequently, binders in the context of the present invention can be not only viscosity-increasing compounds, but also viscosity-reducing compounds. As binders which are preferred, inter alia, mention may be made of, for example, aluminum oxide or aluminum oxide-containing binders, that are described, for example, in WO 94/29408, silicon dioxide as described, for example, in EP 0 592 050 A1, mixtures of silicon dioxide and aluminum oxide as are described, for example, in WO 94/13584, clay minerals, as are described, for example, in JP 03-037156 A, for example montmorillonite, kaolin, bentonite, hallosite, dickite, nacrite and anauxite, alkoxysilanes, as are described, for example, in EP 0 102 544 B1, for example tetraalkoxysilanes such as, for example, tetramethoxysilane, tetraethoxysilane, tetrapropoxysilane, tetrabutoxysilane, or, for example, trialkoxysilanes such as, for example, trimethoxysilane, triethoxysilane, tripropoxysilane, tributoxysilane, alkoxytitanates, for example tetraalkoxytitanates, such as, for example, tetram ethoxytitanate, tetraethoxytitanate, tetrapropoxytitanate, tetrabutoxytitanate, or, for example, trialkoxytitanates, such as, for example, trimethoxytitanate, triethoxytitanate, tripropoxytitanate, tributoxytitanate, alkoxyzirconates, for example tetraalkoxyzirconates, such as, for example, tetramethoxyzirconate, tetraethoxyzirconate, tetrapropoxyzirconate, tetrabutoxyzirconate, or, for example, trialkoxyzirconates such as, for example, trimethoxyzirconate, triethoxyzirconate, tripropoxyzirconate, tributoxyzirconate, silica sols, amphiphilic substances and/or graphites. In particular preference is given to graphite.

As viscosity-increasing compound, use can also be made of, for example, if appropriate in addition to the abovementioned compounds, an organic compound and/or a hydrophilic polymer such as, for example, cellulose or a cellulose derivative such as, for example, methylcellulose and/or a polyacrylate and/or a polymethacrylate and/or a polyvinyl alcohol and/or a polyvinylpyrrolidone and/or a polyisobutene and/or a polytetrahydrofuran.

As pasting agent, use can be made of, inter alia, preferably water or at least one alcohol such as, for example, a monohydric alcohol having 1 to 4 carbon atoms such as, for example, methanol, ethanol, n-propanol, isopropanol, 1-butanol, 2-butanol, 2-methyl-1-propanol or 2-methyl-2-propanol or a mixture of water and at least one of said alcohols or a polyhydric alcohol such as, for example, a glycol, preferably a water-miscible polyhydric alcohol, alone or a mixture with water and/or at least one of said monohydric alcohols.

Further additives which can be used for the kneading and/or shaping are, inter alia, amines or amine derivatives such as, for example, tetraalkylammonium compounds or aminoalcohols and carbonate-containing compounds such as calcium carbonate. Such further additives are described, for instance in EP 0 389 041 A1, EP 0 200 260 A1 or WO 95/19222.

The sequence of the additives such as template compound, binder, pasting agent, viscosity-increasing substance in the shaping and kneading is in principle not critical.

According to a further preferred embodiment, the shaped body obtained according to kneading and/or shaping is subjected to at least one drying, which is generally carried out at a temperature in the range from 25 to 300° C., preferably in the range from 50 to 300° C., and particularly preferably in the range from 100 to 300° C. Likewise, it is possible to dry in a vacuum or under a protective gas atmosphere or by spray drying.

According to a particularly preferred embodiment, in the context of this drying operation, at least one of the compounds added as additives is at least in part removed from the shaped body.

### EXAMPLE

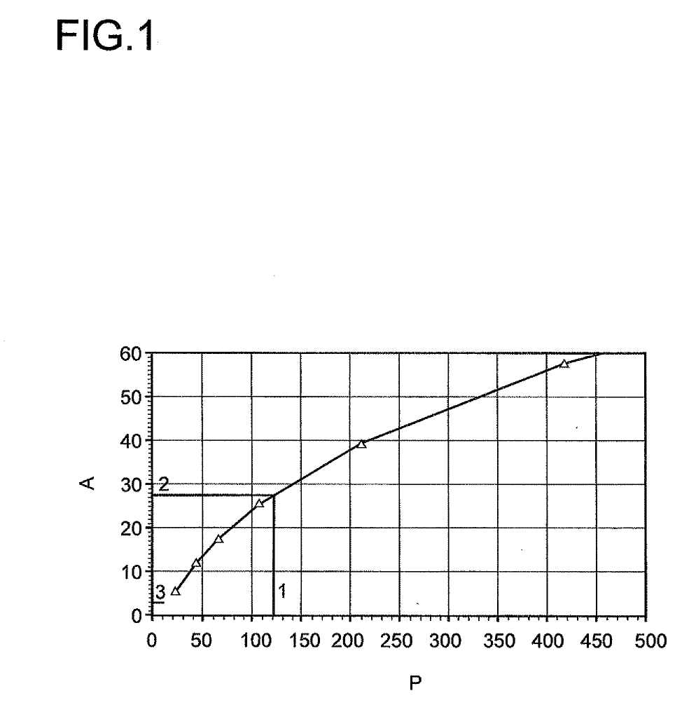

FIG. 1 shows the adsorption isotherms of CO₂ at 20° C. to the metal-organic framework material aluminum terephthalate in the form of 3×3 mm tablets, P giving the absolute pressure in mbar and A giving the amount of adsorbed gas in mg per g of adsorbent.

At an exemplary diving depth of 20 m (3 bar), the partial pressure of CO₂ (4%×3 bar) is 120 mbar (see point 1 in FIG. 1). This corresponds to a loading of 24 to 27 mg/g (see point 2 in FIG. 1).

At the surface, for example in the dive center, fresh air is blown through the adsorber bed in countercurrent flow using the conventional compressor. The CO₂ partial pressure is (0.03%×1 bar) 0.3 mbar (see point 3 in FIG. 1).

This means that 1 mol of CO₂ can be adsorbed using about 2 kg of framework material.

1-13. (canceled)

14. A method for removing carbon dioxide and optionally water from breathing air in closed or partially closed systems comprising
contacting the breathing air with a porous metal-organic framework material, the framework material comprising at least one at least bidentate organic compound which is bound by coordination to at least one metal ion, wherein the closed or partially closed system comprises at least one breathing apparatus and also a breathing mask, a breathing suit or other life support systems.

15. The method according to claim 14, wherein the porous metal-organic framework material is present as a shaped body.

16. The method according to claim 14, wherein the porous metal-organic framework material is at least part of an adsorber bed in a filter.

17. The method according to claim 14, wherein the at least one metal ion is an ion selected from the group consisting of aluminum, zinc and copper.

18. The method according to claim 16, wherein the at least one metal ion is an ion selected from the group consisting of aluminum, zinc and copper.

19. The method according to claim 14, wherein the at least bidentate organic compound is derived from a dicarboxylic acid, tricarboxylic acid, tetracarboxylic acid, or their sulfur analogs.

20. The method according to claim 18, wherein the at least bidentate organic compound is derived from a dicarboxylic acid, tricarboxylic acid, tetracarboxylic acid, or their sulfur analogs.

21. A closed or partially closed system which comprises at least one breathing apparatus and also a breathing mask, a breathing suit or other life support system, in addition, comprising a porous metal-organic framework material, the framework material comprising at least one, at least bidentate, organic compound which is bound by coordination to at least one metal ion.

22. The closed or partially closed system according to claim 21, wherein this in addition has a filter, in which the porous metal-organic framework material is present at least as part of an adsorber bed.

23. The closed or partially closed system according to claim 22, wherein the filter is exchangeable or is installed fixed in the system.

24. A method for regenerating a porous metal-organic framework material from a closed or partially closed system according to claim 21 comprising the steps
optionally removing the metal-organic framework material; and
impinging the framework material with a gas.

25. The method according to claim 24, wherein the gas is air, nitrogen, an inert gas, or a mixture thereof.

26. The method according to claim 24, wherein the impingement is together with the change of at least one parameter selected from pressure and temperature.

27. The method according to claim 24, wherein the regeneration of the metal-organic framework material is performed during the use of the closed or partially closed system which comprises at least one breathing apparatus and also a breathing mask, a breathing suit or other life support system, in addition, comprising a porous metal-organic framework material, the framework material comprising at least one, at least bidentate, organic compound which is bound by coordination to at least one metal ion.
